(19) United States
(12) Patent Application Publication
(10) Pub. No.: US 2005/0031652 A1
(43) Pub. Date: Feb. 10, 2005

(54) Compositions and methods comprising memantine and polyanionic polymers

(75) Inventors: Patrick M. Hughes, Aliso Viejo, CA (US); Orest Olejnik, Coto de Caza, CA (US); Rhett Schiffman, Laguna Beach, CA (US)

Correspondence Address: Frank J. Uxa;Stout, Uxa, Buyan & Mullins, LLP, Suite 300, 4 Venture, Irvine, CA, 92618, US

(73) Assignee: Allergan, Inc., Irvine, CA (US)

(21) Appl. No.: 10/941,272

(22) Filed: Sep. 14, 2004

Related U.S. Application Data
(63) Continuation-in-part of application No. 10/752,125, filed on Jan. 5, 2004.

(30) Foreign Application Priority Data
Feb. 25, 2003 (DE) 103 07 856.8

(51) Int. Cl. A61K 31/13

(52) U.S. Cl. 424/400; 514/662

(57) ABSTRACT

Compositions are provided including a neuroprotective amine related to adamantane and a polyanionic polymer which are well tolerated, non-toxic and/or result in reduced or fewer side effects. Methods are provided employing such compositions, for example, topically administering such compositions to human or animal eyes, for treating human or animal eyes.

### RELATED APPLICATION

This application is a continuation-in-part of application Serial No. 10/752,125 filed Jan. 5, 2004, which is incorporated in its entirety herein by reference.

### BACKGROUND OF THE INVENTION

The present invention is directed to compositions and methods for treating eyes. More particularly, the invention relates to such compositions including a neuroprotective component, and to such methods involving administering a neuroprotective component.

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupilary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe, and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. Several topical ophthalmic therapeutic agents are currently administered to patients in an effort to reduce intraocular pressure, including prostaglandins and prostamides, alpha-2 adrenergic agonists, alpha-2 adrenergic antagonists, and others.

In addition to intraocular pressure reduction, a complimentary approach to the treatment of the sequelae of glaucoma is the administration of neuroprotective agents. Glaucoma is associated with an increase in the rate of retinal ganglion cell loss, resulting in vision loss. U.S. Pat. No. 6,482,854 and Sugrue (Journal of Medicinal Chemistry, 1997, Vol. 40, No. 18, 2793-2809) teach the use of neuroprotective agents to treat glaucoma. While the exact mechanism of these neuroprotective agents may not be unambiguously established, it is believed that these compounds work as glutamate antagonists. Retinal ganglion cells, like other ganglion cells, have surface receptors for glutamate as well as other amino acids, which trigger neuronal excitation. However, excess amino acid associated neuroexcitation causes neuronal degeneration and cell death. In the case of glaucoma, vitreous concentrations of glutamate are double that of a healthy individual, and thus it is believed that the excess glutamate causes accelerated ganglion cell loss and accompanying loss of vision. There are several types of glutamate receptors which are classified based on their function and mechanism of action. One class of glutamate receptors, the ionotropic receptors, works through Ca²⁺-specific ion channels. This class can be divided into subclasses based upon their selective agonists. It is believed that memantine and other adamantane-based amines act as antagonists to one of these subclasses of receptors, referred to as the N-methyl-D-aspartate (NMDA) receptor according to the name of its selective agonist. Thus, memantine and other adamantane-based amines counteract glutamate neuroexcitotocity, and retard vision loss in glaucoma sufferers.

In addition to the treatment of glaucoma, it is believed that memantine and other adamantane-based glutamate antagonists are useful in the treatment of other diseases. U.S. Pat. No. 6,573,280 and U.S. Pat. No. 5,922,773 incorporated herein by reference, teach that glutamate causes migration and proliferation of retinal pigment epithelium and/or glial cells, and is thus useful in treating proliferative vitreoretinopathy.

Topical administration of neuroprotective agents can result in irritation and the like disadvantageous effects to the eye. Thus, compositions for topical administration of neuroprotective agents often have reduced concentrations of such agents to mitigate against such effects. However, reduced concentrations of the neuroprotective agents can disadvantageously result in reduced therapeutic efficacy in the eye.

Oral dosing of neuroprotective agents is a useful approach to providing such agents to the eye, in particular, the posterior segment of the eye, such as the retina, as well as to the optic nerve. Such oral dosing has some issues. For example, oral dosing of neuroprotective agents may need to proceed for a prolonged period of time, for example, days or even weeks, before such dosing provides a therapeutically effective amount of the agent in the posterior segment of the eye. Thus, oral dosing is less than ideal for the treatment of acute conditions, such as retinol detachment, retinal vascular occlusions and the like, as well as for laser induced damage prophylaxis. Further, oral dosing of neuroprotective agents may be accompanied by side effects, such as dizziness, lightheadedness, slurred speech and the like. In certain situations, oral doses are reduced to avoid these side effects, which prolongs the period before the agent is therapeutically effective in the posterior segment of the eye.

It would be advantageous to provide compositions and methods which are safe and effective and mitigate against one or more of the above-noted problems or issues.

### SUMMARY OF THE INVENTION

New compositions and methods for providing neuroprotective components to eyes have been discovered. The present compositions, when topically administered to an eye, are tolerated by the eye and/or are non-toxic to the eye. Thus, the present compositions provide the desired therapeutic effect to the eye without causing undue harm, for example, being substantially non-irritating, to the eye. Moreover, it has been found that the present compositions, useful for topical administration to the eye, provide substantial advantages over oral dose compositions, for example, in terms of reduced or fewer side effects, and an ability to more quickly provide a therapeutically effective amount of the neuroprotective agent to the eye, in particular the posterior segment of the eye. The present methods involve topically administering a composition, for example, a composition in accordance with the present invention, including a neuroprotective component to an eye. The present compositions and methods are relatively straightforward and easy to produce, use and practice. In short, the present compositions and methods substantially increase the usefulness of neuroprotective agents for the treatment of ophthalmic conditions.

In one broad aspect of the present invention, compositions are provided comprising an aqueous-based carrier and more than 0.1% w/v of an adamantane-based neuroprotective component solubilized in the carrier. The composition is such that, when the composition is topically administered to an eye, the composition is at least one of tolerated by the eye, non-toxic to the eye and effective to provide fewer side effects and/or reduced side effects relative to orally administering the neuroprotective component. Preferably the topically administered composition is both tolerated by the eye and non-toxic to the eye.

In a useful embodiment, the composition, when topically administered to the eye is substantially non-irritating to the eye and/or causes substantially no discomfort and/or causes substantially no pain. The compositions, when topically administered to an eye, are more beneficial than detrimental to the eye, and advantageously cause substantially no interference with at least one, and more preferably both, of the appearance of the eye and the functioning of the eye.

In another broad aspect of the present invention, methods for treating an eye of a human or animal are provided. These methods comprise topically administering to an eye of a human or animal a composition comprising an aqueous-based carrier and an adamantane-based neuroprotective component solubilized in the carrier to provide a concentration of the neuroprotective component in the retina of the eye of at least about 0.3 μM or about 0.4 μM, and at least one of fewer side effects and reduced side effects relative to orally administering the adamantane-based neuroprotective component to the human or animal to provide the same concentration of the neuroprotective component in the retina of the eye.

In one useful embodiment, the administering step provides a concentration of neuroprotective component in the retina of the eye of at least about 0.5 μM. Advantageously, the administering step provides fewer side effects and reduced side effects relative to orally administering the adamantane-based neuroprotective component to the human or animal to provide the same concentration of the neuroprotective component in the retina of the eye.

The present compositions, as described herein, can be, and preferably are, used in the present methods.

In a further broad aspect of the present invention, methods for treating an eye of a human or animal are provided which comprise topically administering a first composition to an eye of a human or animal, and orally administering a second composition to a human or animal. The first composition comprises an aqueous-based carrier and a first adamantane-based neuroprotective component solubulized in the carrier. The second composition comprises a second adamantane-based neuroprotective component. Advantageously the orally administering step occurs during and/or after, more preferably after, the topically administering step. In one useful embodiment, the topically administering step provides a therapeutically effective amount of an adamantane-based neuroprotective component to a retina of the eye more rapidly than an identical method without the topically administering step.

The topically administering step provides a concentration of at least about 0.3 μM or at least about 0.4 μM, and more preferably, at least about 0.5 μM of the adamantane-based neuroprotective component in a retina of an eye.

The present methods may be employed to treat acute indications, for example, an acute retinal injury, chronic indications, or as a prophylaxis for the eye.

The topically administering step advantageously provides a greater retinal concentration of an adamantane-based neuroprotective component than the orally administering step.

The first and second adamantane-based neuroprotective components may be the same or different.

The adamantane-based neuroprotective components useful in the present invention preferably comprise an adamantyl moiety and an amine moiety, for example, with the adamantyl moiety bonded directly or indirectly to a nitrogen of the amine moiety. A linking group may be provided which is bonded to both the adamantyl moiety and the amine moiety.

The adamantyl moiety may include no substituent, or one substituent or a plurality of substituents. The amine moiety may be selected from a primary amine moiety, a secondary amine moiety and a tertiary amine moiety.

In one embodiment, the adamantane-based neuroprotective component is effective, when the present composition is topically administered to an eye, to reduce the rate of ganglion cell loss as a result of a neurodegenerative disease in an eye.

The adamantane-based neuroprotective component advantageously is selected from amanitadine, remantadine, memantine, salts thereof and mixtures thereof, more preferably memantine, salts thereof and mixtures thereof.

The adamantane-based neuroprotective component may be present in the present compositions in a wide range of concentrations. Examples of such useful concentrations range from about 0.01% (w/v) or about 0.1% (w/v) or more than 0.1% (w/v) to about 0.5% (w/v) or about 1.0% (w/v) or about 1.5% (w/v) or about 3% (w/v) or about 5% (w/v) or more.

In a particularly useful embodiment, the present compositions further comprise a water soluble polymeric compatibility component present in an amount effective to enhance the ocular compatibility of the adamantane-based neuroprotective component relative to an identical composition without the compatibility component. Advantageously, the compatibility component is a polyanionic polymeric component.

The compatibility component may be present in the presently useful compositions in a wide variety of concentrations, provided that such component functions as a compatibility component and does not have an undue detrimental effect on the remainder of the composition, on the functioning of the composition or on the eye or functioning of the eye. For example, the compatibility component may be present in an amount in the range of about 0.01% (w/v) or about 0.1% (w/v) to about 5% (w/v) or about 8% (w/v) or about 10% (w/v).

Any suitable compatibility component may be employed provided it functions as a compatibility component and has no undue or significant detrimental effect on the composition or on the eye being treated. In one embodiment, the compatibility component is selected from anionic cellulosic derivatives, hyaluronic acid, anionic starch derivatives, poly methacrylic acid, poly methacrylic acid derivatives, polyphospazene derivatives, poly aspartic acid, gelatin, alginic acid, alginic acid derivatives, poly acrylic acid, poly acrylic acid derivatives, and the like and mixtures thereof. Very useful compatibility components include anionic cellulosic derivatives and mixtures thereof, especially carboxymethyl cellulose.

Each and every feature described herein, and each and every combination of two or more of such feature, is included within the scope of the present invention provided that the features included in such a combination are not mutually inconsistent.

These and other aspects and advantages of the present invention are apparent in the following detailed description, drawing examples and claims, particularly when considered in conjunction with the accompanying drawing.

### BRIEF DESCRIPTION OF THE DRAWING

FIG. 1 is a plot of the permeability of carboxymethylcellulose (CMC) and non-CMC formulations of memantine through dialysis membranes.

### DETAILED DESCRIPTION OF THE INVENTION

Compositions and methods are provided which involve adamantane-based neuroprotective components. Such compositions, when topically administered to an eye, are tolerated by the eye and/or are non-toxic to the eye and/or provide reduced and/or fewer side effects relative to oral administration of the neuroprotective components.

In one embodiment, the present compositions, such as in the form of solutions, comprise an aqueous-based carrier and an adamantane-based neuroprotective component, for example, more than 0.1% (w/v) of an adamantane-based neuroprotective component. In a very useful embodiment, the compositions further comprise a water soluble compatibility component, for example, a polyanionic polymeric compatibility component, present in an amount effective to enhance the ocular compatibility of the adamantane-based neuroprotective component or compositions relative to an identical composition without the compatibility component.

An adamantane-based neuroprotective component comprises an adamantane-based amine. Such a compound includes an amine or amino group which is directly or indirectly bonded or coupled to adamantane, which has the following structure: 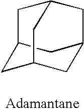
In other words, adamantane may be directly bonded to the nitrogen of the amine, or a linking group consisting of one or more atoms may connect the adamantane to the amine. The adamantane may have one or more additional substituents, such as a methyl group or other small or lower, for example, containing about 4 or less carbon atoms, alkyl group, attached. A reactable group or moiety comprising the basic cage structure of adamantane, with or without one or more substituents, is referred to herein as an “adamantyl” moiety. The term “amine” should be understood as being broadly applied to both a molecule, or a moiety or functional group, as generally understood in the art, and may be a primary amine, a secondary amine, or a tertiary amine.

A neuroprotective component is a substance, e.g., compound, mixture of compounds, other composition, other material and the like, which is generally understood in the art to reduce the rate of ganglion cell loss in or as a result of a neurodegenerative disease or condition, such as Alzheimer's disease, glaucoma and the like.

While not intending to limit the scope of the invention in any way, three compounds which are adamantane-based amines, and are also neuroprotective components or compounds comprising an adamantyl moiety and an amine moiety, are amantadine, rimantadine, and memantine, which have the following structures.

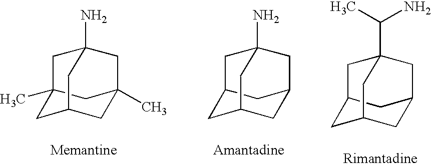

Memantine Amantadine Rimantadine

The terms “memantine”, “amantadine”, and “rimantadine” as used herein refer to the free base forms of the amine, and any one or more various pharmaceutically acceptable salts thereof, such as memantine hydrochloride and the like, which can be prepared by the addition of an acid to the free base.

The determination of the amount of adamantane-based neuroprotective component, for example, memantine, used in the compositions disclosed herein is well within the ability of one having ordinary skill in the art. An “effective” amount of adamantane-based neuroprotective component, for example, memantine, in a composition is an amount, when the composition is administered to a human or animal, which has a detectable effect, for example, a detectable neuroprotective effect, relative to an identical composition without the adamantane-based neuroprotective component.

In referring to concentrations of adamantane-based neuroprotective component, for example, memantine, herein, the numeric value for the concentration is understood to be the concentration of the free base, regardless of the form in which the adamantane-based neuroprotective component is used. Since there is a large range of concentrations or amounts at which the adamantane-based neuroprotective component is effective, the concentration or amount of the adamantane-based neuroprotective component in the presently useful compositions may vary over a relatively wide range.

In one embodiment, the present compositions include more than 0.1% (w/v) of an adamantane-based neuroprotective component solubilized in the carrier. In a useful embodiment, the present compositions comprise from about 0.05% (w/v) to about 2% (w/v), or about 2.5% (w/v) or about 5% (w/v) of the adamantane-based neuroprotective component, for example and without limitation, memantine. Other useful compositions comprise from about 0.2% (w/v) to 3% (w/v), or about 0.1% (w/v) to about 2% (w/v), or about 0.5% (w/v) to about 2% (w/v) of the adamantane-based neuroprotective component. Still further useful compositions comprise about 0.5% (w/v) to about 3.5% (w/v), or about 0.3% (w/v) to about 1.5% (w/v), or about 0.5% (w/v) to about 1.3% (w/v), or about 0.1% (w/v) to about 1% (w/v), or about 0.5% (w/v) to about 1% (w/v), or about 0.5% (w/v) or about 1% (w/v) of an adamantane-based neuroprotective component.

In one aspect of the present invention, the compositions comprise an aqueous-based carrier and more than 0.1% w/v if the adamantane-based neuroprotective component solubilized in the carrier. Such compositions, when topically administered to an eye, are tolerated by the eye and/or non-toxic to the eye.

As noted above, the present compositions may include a compatibility component.

Although any suitable and useful compatibility component may be employed in accordance with the present invention, in one very useful embodiment, the compatibility component is a water soluble polymeric component, and advantageously a water soluble polyanionic polymeric component.

The term “polyanionic polymeric component” or “polyanionic polymer” refers, in the broadest sense understood in the art, to a polymeric material or polymer comprising a plurality of, for example, several, anionic moieties per molecule. While not intending to limit the scope of the invention in any way, typical examples of polyanionic polymers include anionic cellulosic derivatives, such as carboxymethylcellulose and the like, hyaluronic acid, anionic starch derivatives, such as carboxymethylamylose and the like, anionic polymers derived from acrylic acid (meaning to include polymers from acrylic acid, acrylates, and the like and mixtures thereof), anionic polymers derived from methacrylic acid (meaning to include polymers from methacrylic acid, methacrylates, and the like and mixtures thereof), poly(methacrylic acid) derivatives, polyphospazene derivatives, poly(aspartic acid), anionic polymers of amino acids (meaning to include polymers of amino acids, amino acid salts, and the like and mixtures thereof), acidic gelatin, and anionic polymers derived from alginic acid (meaning to include alginic acid, alginates, and the like and mixtures thereof). In one embodiment, the polyanionic polymeric component comprises carboxymethylcellulose. Carboxymethylcellulose is a polyanionic species, and thus may have one or more counter or countering cations, by which it may be referred. For example, sodium carboxymethylcellulose refers to carboxymethylcellulose having sodium as the counter ion.

The term “soluble”, in reference to a polymeric compatibility component or polyanionic polymeric component, means that the component or polymer dissolves in an aqueous solution, for example an aqueous-based carrier, at an effective concentration.

An “effective” amount or concentration of a polymeric compatibility component or polyanionic polymeric component is an amount which provides a composition with a detectable compatibility effect relative to an identical composition without the polymeric compatibility component or polyanionid polymeric component. Since there is a relatively large range of concentrations or amounts of the polymeric compatibility component or polyanionic polymeric component that are effective, the concentration or amount of such component may vary over a relatively wide range in the compositions and methods disclosed herein.

In one embodiment, the present compositions include a compatibility component in an amount in a range of about 0.1% (w/v) to about 5% (w/v) or about 8% (w/v) or about 10% (w/v) of the total composition. Examples of useful compositions comprise about 0.1% (w/v) or about 0.3% (w/v) or about 0.4% (w/v) or about 0.5% (w/v) to about 0.8% (w/v) or about 1% (w/v) or about 1.5% (w/v) or about 2% (w/v) or about 4% (w/v) or about 4.5% (w/v) or about 5% of a polyanionic polymeric component, for example, carboxymethylcellulose, such as sodium carboxymethylcellulose. For example, the present compositions may include about 0.5% (w/v) sodium carboxymethylcellulose.

Preservative components may be used to prevent microbial contamination in multiple-use ophthalmic preparations. Cationic, anionic, and nonionic preservatives may be used. Examples, without limitation, of useful preservative components include benzalkonium chloride, stabilized oxychloro complexes or stabilized chlorine dioxide, for example, identified as a product by the trademark Purite®, owned by Allergan, Inc., phenylmercuric acetate, chlorobutanol, benzyl alcohol, parabens, thimerosal and the like and mixtures thereof.

Certain compositions disclosed herein may comprise a cationic preservative. While not intending to limit the scope of the invention, or be bound in any way by theory, it is generally expected that cationic preservatives will form an insoluble complex with the polyanionic polymeric component which complex precipitates from solution. However, the compositions prepared in Example 1, set forth hereinafter, contain a cationic preservative, and no insoluble material formed during or subsequent to formation of the compositions. Thus, while not intending to limit the scope of the invention, the compositions disclosed herein may have an added advantage of flexibility in terms of the use of preservatives. Quaternary ammonium salts, such as benzalkonium chloride, are common cationic preservatives which may be employed.

An “effective” amount or concentration of a preservative is the concentration required to significantly inhibit the growth of microbes in a composition, relative to an identical or similar composition without the preservative component. Since there are a relatively large range of concentrations or amounts of the preservative component that are effective, the concentration or amount of such component may vary over a relatively wide range in the compositions and methods disclosed herein. An effective amount or concentration encompasses a large range of values, the concentration or amount of the cationic preservative used herein may vary significantly. In one embodiment, from about 10 ppm to about 200 ppm of preservative component, for example, benzalkonium chloride is used. Another composition comprises about 20 ppm of preservative component, for example, benzalkonium chloride. In another embodiment, the present compositions may be free of components effective as preservative components.

In addition to being useful in the treatment of glaucoma, the present compositions can be used to reduce or control retinal pigment epithelium and/or glial migration and the diseases or conditions related thereto. Thus, the compositions disclosed herein can be used to treat diseases or conditions wherein migration or proliferation of retinal pigment epithelium or glial cells cause or contribute to the cause of such diseases or conditions. The relationship may be direct or indirect, and the migration or proliferation of retinal pigment epithelium or glial cells may be a root cause of the disease or condition, or may be a symptom of another underlying disease or condition.

While not intending to limit the scope of the invention in any way, the following are examples of the types of diseases or conditions that can be treated using the present compositions and/or methods: non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, retinitis pigmentosa, and the like diseases and conditions.

Other specific embodiments are contemplated herein, which are combinations of the aforementioned embodiments. Those skilled in the art will also recognize that additional embodiments may also be prepared by combining the aforementioned embodiments, which would also be considered to be within the scope and spirit of the present invention.

While not intending to limit the scope of the invention in any way, it is often useful to include a buffer component in ophthalmic compositions in an amount effective to maintain the pH of the present composition in a desired, physicologically acceptable range, for example, in a range of about 6 to about 8. Compositions having such pH's provide substantial advantages including but not limited to, patient comfort.

Buffer components useful in accordance with the present invention include, without limitation, those known as useful in pharmaceutical, e.g., ophthalmic compositions to those skilled in the art. Some examples of such buffer components include, without limitation, acetate, borate, carbonate, citrate, phosphate and the like buffers and mixtures thereof.

Tonicity components may be included in the present compositions in amounts effective to provide such compositions with a desired tonicity, for example, without limitation, to provide substantially isotonic composition. Examples of useful tonicity components include, without limitation, glycerin, mannitol, sorbitol, sodium chloride, potassium chloride and the like and mixtures thereof.

Surfactant components may be employed, in effective concentrations or amounts in the present compositions. Examples of useful surfactant components include, without limitation, polysorbates, poloxamers, alcohol ethoxylates, ethylene glycol-propylene glycol block copolymers, fatty acid amides, alkylphenol ethoxylates, phospholipids, and the like and mixtures thereof.

Chelating components may be employed, in effective concentrations or amounts in the present compositions. Examples of useful surfactant components include, without limitation, edetate salts, like edetate disodium, edetate calcium disodium, edetate sodium, edetate trisodium, and edetate dipotassium, and the like and mixtures thereof. The chelating component may be present in an amount effective to enhance preservative effectiveness.

The foregoing discussion of components typically used in ophthalmic compositions is given purely for purposes of example, to more readily enable a person of ordinary skill in the art to carry out the methods disclosed herein, and is not intended to limit the scope of the invention in any way.

In one embodiment of the invention, methods of treating human or animal eyes are provided. Such methods comprise topically administering to an eye of a human or animal a composition comprising an aqueous-based carrier and an adamantane-based neuroprotective component solubilized in the carrier.

Such administering is advantageously effective to provide a concentration of the neuroprotective component in the retina of the eye of at least about 0.3 μM or at least about 0.4 μM or at least about 0.5 μM. Compositions in accordance with the present invention may be, and preferably are, used in such methods.

This topically administering step is tolerated by the eye and/or is non toxic to the eye and/or is effective to provide at least one of fewer side effects and reduced side effects relative to orally administering the adamantane-based neuroprotective component to the human or animal to provide the same retinal concentration of the neuroprotective component.

It has been found that topical dosing of adamantane-based neuroprotective component, for example, memantine, in accordance with the present invention provides a therapeutic concentration or amount in the optic nerve of the eye being treated. Such topical administration of memantine can rapidly achieve equivalent or greater retinal levels of such neuroprotective component relative to oral delivery. Moreover, due to the side effects limitation of oral dosing, topical dosing can actually achieve higher retinal concentrations than those achieved orally, for example, with fewer and/or reduced side effects.

Oral delivery of adamantane-based neuroprotective components, such as memantine, is associated with a number of dose limiting side effects including: dizziness, lightheadedness and slurred speech. Other adverse events associated with CNS excitation include anxiety, dissociation, dry mouth, headache, nervousness, slurred speech, tiredness and weakness and occur from memantine oral dosages of 5 to 30 mg/day. Topical administration of these component, in accordance with the present invention, can achieve much higher ocular levels of adamantane-based neuroprotective component, for example, memantine, while greatly reducing the systemic exposure to such component. Moreover, the low systemic exposure means that the dose ramp from 5 mg to 20 mg required for oral delivery would not be required for topical administration. This is especially relevant for acute indications, such as retinal detachment, damage or injury prophylaxis, for example, laser induced damage prophylaxis, retinal vascular occlusions and the like.

In accordance with the present invention, adamantane-based neuroprotective components can be used topically, for example, as a loading dose, for acute retinal injury, thereby obviating the need to ramp up the dose orally. A substantial medical benefit is realized in that therapeutic concentrations are available substantially immediately without unacceptable systemic adverse events, rather than after a ramp up period.

Thus, in one embodiment, a combination treatment comprising both topically administering a first adamantane-based neuroprotective component to an eye of a human or animal and orally administering a second adamantane-based neuroprotective component to the human or animal is within the scope of the present invention. The first and second adamantane-based neuroprotective components may be the same or different. Advantageously, the topically administering step occurs prior to the orally administering step. For example, the topically administering step may be used to provide a rapid therapeutically effective concentration of the adamantane-based neuroprotective component to the retina or other posterior portion of the eye, for example, as a laser induced damage prophylaxis or because of another acute indication. Once a therapeutic concentration has been achieved, oral administering of an adamantane-based neuroprotective component may be employed, alone or in combination with continued topical administering, to maintain a desired therapeutic concentration.

Oral dosage forms of adamantane-based neuroprotective components are conventional and well known in the art.

The following non-limiting examples illustrate certain aspects and features of the present invention.

### EXAMPLE 1

Memantine, 1-amino-3,5-dimethyladamantane, hydrochloride (memantine HCl), was formulated in a standard aqueous vehicle and a vehicle containing 0.5% sodium carboxymethylcellulose (CMC) (Aqualon Type 7LFH, MW 90 kDa) with 20 ppm benzalkonium chloride (BAK) (Tables 1 and 2).

The compositions of Table 1 were prepared by methods commonly used in the art.

TABLE 1
Non-CMC Memantine HCl Formulations

| Components | Function | Percent w/v | | | | | |
|---|---|---|---|---|---|---|---|
| Memantine | Active | 0.05 | 0.1 | 0.2 | 0.5 | 0.75 | 1.0 |
| HCl | | | | | | | |
| Sodium | Tonicity | 0.46 | 0.46 | 0.46 | 0.36 | 0.30 | 0.23 |
| Chloride | adjuster | | | | | | |
| Boric Acid | Buffering | 0.64 | 0.64 | 0.64 | 0.64 | 0.64 | 0.64 |
| | agent | | | | | | |
| Sodium | Buffering | 0.16 | 0.16 | 0.16 | 0.16 | 0.16 | 0.16 |
| Borate, | agent | | | | | | |
| Decahydrate | | | | | | | |
| Benzalkonium | Preservative | 0.002 | 0.002 | 0.002 | 0.002 | 0.002 | 0.002 |
| Chloride | | | | | | | |
| Hydrochloric | pH | 7.4 | 7.4 | 7.4 | 7.4 | 7.4 | 7.4 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified | Vehicle | Q.S. | Q.S. | Q.S. | Q.S. | Q.S. | Q.S. |
| Water | | | | | | | |

The compositions of Table 2 were prepared according to the following procedure. Two aqueous phases designated Part I and Part II respectively, were separately prepared.

Part I

Purified water (2000 mL) was charged to a vessel and mixing was initiated, and sodium carboxymethylcellulose (CMC) (20 g) was then added and mixed until dispersed.

Part II

Purified water (1700 mL) was charged a vessel and mixing is initiated. Sodium chloride (20.0 g), potassium chloride (5.6 g), sodium lactate (20 ml, 60% solution), calcium chloride (0.80 g), magnesium chloride (0.24 g), and benzalkonium chloride (20 mL) were sequentially added allowing each to dissolve before adding the next. Memantine HCl (4.0 g for the 0.1% w/v formulation) was then added with mixing until dissolved.

After the two aqueous phases were prepared, (Part II) was transferred into the bulk phase (Part I) in the main batch vessel while mixing, and the mixture was thoroughly mixed for 15 minutes. Sodium hydroxide or hydrochloric acid was used to adjust the pH to 6.4-6.6. Water was then added to bring the batch volume to 4000 ml and the pH was adjusted to 6.4-6.6 with 1 N NaOH or 1 N HCl if necessary. The solution was then mixed thoroughly for 20 to 30 minutes, and sterile filtered with a Suporlife DCF CHS92DSPPK 0.2 μm filter. A 500 ml filter flush of the Memantine HCl Topical Solution was required.

TABLE 2
CMC-Memantine HCl Formulations

| Components | Function | Percent w/v | | | | |
|---|---|---|---|---|---|---|
| Memantine | Active | 0.1 | 0.2 | 0.5 | 0.75 | 1.0 |
| HCl | | | | | | |
| Sodium | Tonicity | 0.50 | 0.48 | 0.40 | 0.33 | 0.26 |
| Chloride | adjuster | | | | | |
| Potassium | Electrolyte | 0.14 | 0.14 | 0.14 | 0.14 | 0.14 |
| Chloride | | | | | | |
| Sodium | Electrolyte | 0.3 | 0.3 | 0.3 | 0.3 | 0.3 |
| Lactate | | | | | | |
| Calcium | Electrolyte | 0.02 | 0.02 | 0.02 | 0.02 | 0.02 |
| Chloride, | | | | | | |
| dihydrate | | | | | | |
| Magnesium | Electrolyte | 0.006 | 0.006 | 0.006 | 0.006 | 0.006 |
| Chloride, | | | | | | |
| hexahydrate | | | | | | |
| Benzalkonium | Preservative | 0.002 | 0.002 | 0.002 | 0.002 | 0.002 |
| Chloride | | | | | | |
| Sodium CMC | Compatibility | 0.5 | 0.5 | 0.5 | 0.5 | 0.5 |
| (Type 7LFH) | component | | | | | |
| Hydrochloric | pH adjustment | 6.5 | 6.5 | 6.5 | 6.5 | 6.5 |
| Acid | | | | | | |
| Sodium | pH adjustment | 6.5 | 6.5 | 6.5 | 6.5 | 6.5 |
| Hydroxide | | | | | | |
| Purified | Vehicle | Q.S. | Q.S. | Q.S. | Q.S. | Q.S. |
| Water | | | | | | |

The effect of the CMC on the tolerability of an adamantane-based neuroprotective amine was assessed using sodium carboxymethylcellulose (CMC) as the model polyanionic polymer, and memantine hydrochloride (pKa 10.27) as the model neuroprotective adamantane-based amine.

While not intending to limit the scope of the invention, or be bound in any way by theory, it is believed that a weak electrostatic bond is formed between the cationic drug and the polyanionic species. Thus, it is believed that the weak bond improves the ocular tolerability of the drug while having essentially no impact upon its bioavailability. While not intending to be bound in any way by theory, the experimental results provided herein in this and the other examples to be presented hereafter support this hypothesis.

Osmolality and dialysis studies were carried out with the memantine HCl/CMC model system to demonstrate that the CMC does not significantly diminish the bioavailability of the neuroprotective amine. The osmotic pressure of a solution is a colligative property and as such can be a relative measure of free drug. This relationship is given by equation 1
OP_{TC}=ΔCₘₑₘRT, (1)
where OP_{TC} is the theoretical change in osmotic pressure for a given change in memantine concentration, ΔCₘₑₘ, if the individual memantine molecules are free and unbound. R is the universal gas constant and T the temperature in degrees Kelvin. By comparing the osmolality of memantine formulations, CMC containing memantine formulations and their respective placeboes the activity of the memantine can be inferred.

Osmolality measurements were carried out by freezing point depression osmometry. The results are presented in Table 3.

TABLE 3
Osmolality Comparison of non-CMC Formulations
and CMC Formulations
Osmolality Δπ = ΔCₘₑₘRT

| | | CMC | 0.1% | 1.0% | 0.1% Mem | 1.0% Mem |
|---|---|---|---|---|---|---|
| | Placebo | placebo | Mem | Mem | CMC | CMC |
| Osm/Kg | 177 | 190 | 187 | 267 | 198 | 280 |
| Δπ | N/A | 13 | 10 | 90 | 21 | 103 |
| Theory | N/A | N/A | 9.3 | 93 | 23 | 106 |

The placebo establishes a baseline for the osmolality (the total number of particles per mass of solvent) of the solutions being studied, and the CMC placebo is used to establish the contribution of the CMC to the osmolality of the solution. Thus, the difference of 13 Osm/kg between the two solutions is attributed to the CMC.

Comparison of the osmolality of a solution of 0.1% memantine to the placebo reveals that the memantine increases the osmolality of the solution by 10 Osm/kg, which compares well with the theoretical value of 9.3 based on the amount of CMC added and its molecular weight. The sum of the contribution of the CMC (13 Osm/kg) and the memantine (10 Osm/kg) would therefore predict an expected osmolality of about 23 Osm/kg higher than the placebo solution. The actual osmolality of the 0.1% memantine/CMC solution is 21 Osm/kg higher than the placebo, which is not significantly different than the theoretical expectation. This result suggests that the memantine HCl and CMC behave essentially as individual particles, and not as a single complexed entity, in the 0.1% memantine/CMC solution. A similar result can be made by an analogous comparison between the 1% memantine solutions and the placebos. Hence, while not intending to be bound, or limit the scope of the invention in any way by theory, it is not expected that a weak interaction between memantine and CMC will reduce memantine's bioavailability.

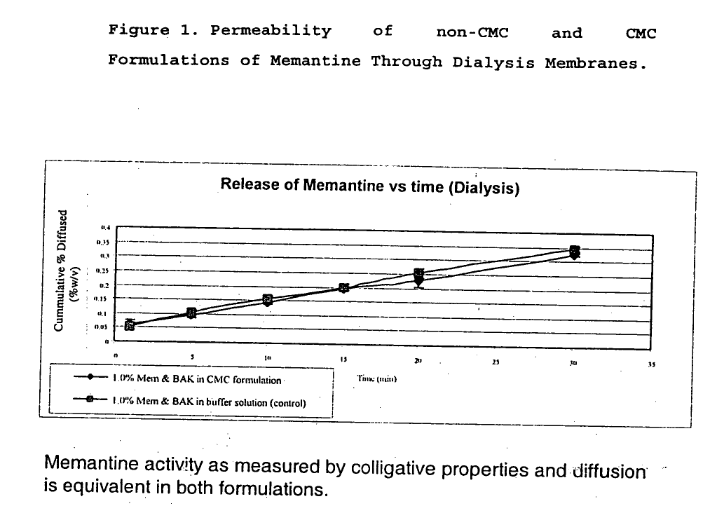

The permeability of memantine through a dialysis membrane was studied as a model for the bioavailability of memantine. If the permeability of memantine through the dialysis membrane is equivalent for the CMC and non-CMC formulations, it is believed that the bioavailability of memantine will not be significantly different for the two types of formulations. These dialysis studies showed that the permeability of memantine from the CMC formulations through the dialysis membrane was equivalent to the permeability of memantine from the borate buffered non-CMC formulation (FIG. 1) through the membrane. In these studies the CMC containing or the non-CMC containing formulations of memantine were placed inside a dialysis bag. The bag was then submerged in a borate buffered formulation placebo reservoir and the appearance of memantine in the reservoir as a function of time was measured. The rate of appearance in the reservoir, drug permeation, is given by equation 2

$$\frac{ⅆ M}{ⅆ t} = \frac{2 π h P}{ln \frac{{r}_{o}}{{r}_{i}}} * {a}_{mem} \qquad ( 2 )$$

where

$$\frac{ⅆ M}{ⅆ t}$$

is the rate of memantine appearance in the reservoir as a function of time, h the thickness of the dialysis bag, P the permeability of memantine in the dialysis membrane, rₒ the outer diameter of the dialysis bag, rᵢ the inner diameter, and aₘₑₘ the memantine activity. In this experiment, only dM/dt and aₘₑₘ are not constant. As such, any difference in the rate of memantine permeation is directly and linearly related to an activity difference. Conversely, if two compositions give similar or identical rates of memantine permeation, the (memantine) activities of those compositions are essentially the same. FIG. 3 clearly shows that the permeation of memantine from non-CMC formulations and CMC formulations is equivalent and as such the activity of memantine is equivalent. While not intending to be bound in any way by theory, or be limited in any way, this suggests that in terms of membrane permeability, memantine activity is essentially the same whether or not a polyanionic polymer is present. Thus, if both the osmolality and activity in terms of membrane permeability are essentially unchanged for memantine in the presence of a polyanionic polymer, it is reasonable to believe that the polyanionic polymer will have a negligible effect on the bioavailability. While not intending to limit the scope of the invention in any way, the bioavailability data presented hereafter supports this conclusion.

### EXAMPLE 2

An initial toxicology screen included borate buffered, isotonic memantine HCl (0.05% to 1.0% w/v) solutions preserved with 20 ppm BAK (Table 1). Additionally, formulations containing 0.5% and 1.0% w/v memantine HCl in 0.5% sodium carboxymethylcellulose (CMC) aqueous vehicle (Table 2) were tested. A one day dose escalation study was conducted to up-titrate to the highest acceptable dose.

Rabbits where dosed with 35 μL of formulation to the cul-de-sac and irritation was ranked as none, slight, mild, moderate or severe. The irritation score was a sum of lacrimation, chemosis, hyperemia and tolerability all scored from none to severe.

The borate buffered placebo showed no irritation. However, the borate buffered memantine formulation showed slight. irritancy at 0.1% (w/v), mild irritancy at 0.5% (w/v) and moderate irritancy at 1.0% (w/v). The CMC placebo showed no irritation and the formulation showed only slight irritation at 1.0% (w/v) memantine. Thus, the CMC formulation has the same irritation as the non-CMC formulation at about a log unit higher memantine concentration. While not intending to be bound or limited in any way by theory, it is unexpected that the CMC would limit the irritation, but have a negligible effect on the predicted bioavailability, as assessed by the dialysis study.

This study was followed up by a five day toxicology study which included borate buffered formulations ranging from 0.1 to 0.75% (w/v) memantine, CMC based formulations ranging from 0.1 to 1.0% (w/v) memantine and the relevant placebos. All formulations were preserved with 20 ppm BAK.

The formulations were dosed with 35 μL drops topically to the cul-de-sac of rabbits twice a day for 1 week. The rabbits were evaluated by gross clinical observation of irritation, ranked as none, slight, mild, moderate or severe. The irritation score was a sum of lacrimation, chemosis, hyperemia and tolerability all scored from none to severe.

The borate buffered memantine formulations displayed a dose dependent increase in severity and frequency of ocular discomfort ranging from slight at 0.1% (w/v) to moderate at 0.75% (w/v) memantine. The CMC based formulations displayed only slight discomfort at 0.5% (w/v) and 0.75% (w/v) memantine, with mild discomfort at 1.0% memantine. Thus, the tolerability of memantine is improved by a factor of about 5-8. Conjunctival hyperemia was slight to mild at 0.5% (w/v) or higher memantine for the borate solution, while a low frequency of slight hyperemia was observed for the CMC based formulation at 0.5% and 1.0% (w/v) memantine, but not the other memantine concentrations. Again, while not intending to be bound by theory, hyperemia is significantly reduced for the formulation containing the polyanionic polymer.

It was also important to assess the impact of the CMC on ocular bioavailability. A pharmacokinetic study was conducted to compare ocular concentrations after 0.05% non-CMC and 0.05% CMC formulations and to assess dose linearity of two CMC formulations (0.05% versus 0.25%). The formulations were topically dosed Female albino rabbits twice a day for 7 days to both eyes. Non-radiolabeled formulations were manufactured and spiked with ¹⁴C-Memantine HCl with a specific activity of 33.7 mCi/mmol. Final activity of the formulations ranged from 66.3 to 72.5 μCi/mL. After dosing the rabbits, six eyes per time point were sampled at predose, 30, 60, 120 and 240 minutes post dose. The conjunctiva, aqueous humor, cornea, iris-ciliary body, lens and sclera were assayed. Vitreous humor, retina and choroid were assayed only at predose, 30, 120 and 240 minutes post-dose. Tissue memantine concentrations were determined by tissue combustion with activity measurement by scintillation counter. The vitreous and aqueous humors were counted without combustion. Disintegrations per minutes were then correlated to tissue concentrations.

At 0.05% w/v memantine, the CMC and the non-CMC formulations displayed equivalent retinal concentrations. Retinal concentrations of the 0.25% memantine/CMC based formulation were approximately 4 fold higher than the retinal concentrations of the 0.05% formulations, thus indicating a nearly linear dose response.

The retina concentrations (Cₘₐₓ) for the 0.05% (w/v) memantine borate buffered formulation, the 0.05% (w/v) memantine/CMC formulation and the 0.25% (w/v) memantine/CMC formulation were 64.6 ng/mL, 63.9 ng/mL and 289 ng/mL, respectively. The higher dose corresponds to 1.4 μM.

The tₘₐₓ in the retina for all topical administrations was 30 minutes, indicating a rapid and immediate dosing of memantine useful/effective for acute indications.

Thus, while not intending to limit the scope of the invention, or be bound in any way by theory, these results show that the irritancy mitigating affect of the CMC on memantine does not significantly reduce the bioavailability. While not intending to be limited or bound in any way by theory, these results indicate that a higher concentration of memantine may be used in a formulation while the irritation is the same or less than that previously observed at lower concentrations. Alternatively, while not intending to be limited in any way by theory, one may formulate a less irritating memantine composition by the addition of a polyanionic polymer.

### EXAMPLE 3

Compositions are prepared according to the procedure of Example 1 using the formulas described in Table 4.

TABLE 4

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.65 | 0.65 | 0.65 | 0.65 | 0.65 | 0.65 |
| Chloride | adjuster | | | | | | |
| Boric Acid | Buffering | 0.64 | 0.64 | 0.64 | 0.64 | 0.64 | 0.64 |
| | agent | | | | | | |
| Sodium | Buffering | 0.16 | 0.16 | 0.16 | 0.16 | 0.16 | 0.16 |
| Borate, | agent | | | | | | |
| Decahydrate | | | | | | | |
| Stabilized | Preservative | 0.005 | 0.005 | 0.005 | 0.005 | 0.005 | 0.005 |
| Oxychloro | | | | | | | |
| Complex | | | | | | | |
| Sodium CMC | Polyanionic | 0.5 | 0.5 | 0.5 | 0.5 | 0.5 | 0.5 |
| (Type 7LFH) | polymer | | | | | | |
| Hydrochloric | pH | 7.4 | 7.4 | 7.4 | 7.4 | 7.4 | 7.4 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified | Vehicle | Q.S. | Q.S. | Q.S. | Q.S. | Q.S. | Q.S. |
| Water | | | | | | | |

### EXAMPLE 4

Compositions are prepared according to the procedure of Example 1 using the formulas described in Table 5.

TABLE 5

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.72 | 0.72 | 0.72 | 0.72 | 0.72 | 0.72 |
| Chloride | adjuster | | | | | | |
| Sodium Citrate | Buffering | 0.45 | 0.45 | 0.45 | 0.45 | 0.45 | 0.45 |
| | agent | | | | | | |
| Citric Acid | Buffering | 0.01 | 0.01 | 0.01 | 0.01 | 0.01 | 0.01 |
| | agent | | | | | | |
| Benzalkonium | Preservative | 0.005 | 0.005 | 0.005 | 0.005 | 0.005 | 0.005 |
| Chloride | | | | | | | |
| Carbomer 940 | Polyanionic | 0.2 | 0.2 | 0.2 | 0.2 | 0.2 | 0.2 |
| | Polymer | | | | | | |
| Hydrochloric | pH | 6.3 | 6.3 | 6.3 | 6.3 | 6.3 | 6.3 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

### EXAMPLE 5

Compositions are prepared according to the procedure of Example 1 using the formulas described in Table 6.

TABLE 6

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.65 | 0.65 | 0.65 | 0.65 | 0.65 | 0.65 |
| Chloride | adjuster | | | | | | |
| Boric Acid | Buffering | 0.64 | 0.64 | 0.64 | 0.64 | 0.64 | 0.64 |
| | agent | | | | | | |
| Sodium Borate, | Buffering | 0.16 | 0.16 | 0.16 | 0.16 | 0.16 | 0.16 |
| Decahydrate | agent | | | | | | |
| Chlorobutanol | Preservative | 0.2 | 0.2 | 0.2 | 0.2 | 0.2 | 0.2 |
| Sodium CMC | Polyanionic | 0.5 | 0.5 | 0.5 | 0.5 | 0.5 | 0.5 |
| (Type 7LFH) | Polymer | | | | | | |
| Hydrochloric | pH | 7.4 | 7.4 | 7.4 | 7.4 | 7.4 | 7.4 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

### EXAMPLE 6

Compositions are prepared according to the procedure of Example 1 using the formulas described in Table 7.

TABLE 7

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.72 | 0.72 | 0.72 | 0.72 | 0.72 | 0.72 |
| Chloride | adjuster | | | | | | |
| Sodium Citrate | Buffering | 0.45 | 0.45 | 0.45 | 0.45 | 0.45 | 0.45 |
| | agent | | | | | | |
| Citric Acid | Buffering | 0.01 | 0.01 | 0.01 | 0.01 | 0.01 | 0.01 |
| | agent | | | | | | |
| Chlorobutanol | Preservative | 0.2 | 0.2 | 0.2 | 0.2 | 0.2 | 0.2 |
| Carbomer 940 | Polyanionic | 0.2 | 0.2 | 0.2 | 0.2 | 0.2 | 0.2 |
| | Polymer | | | | | | |
| Hydrochloric | pH | 6.3 | 6.3 | 6.3 | 6.3 | 6.3 | 6.3 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

### EXAMPLE 7

A polyanionic polymer containing composition according to one of examples 1-6 is administered twice daily to a person suffering from glaucoma for a prolonged period of time. Irritation is below a tolerable level throughout the treatment, and the rate of vision loss is significantly reduced.

### EXAMPLE 8

General disposition of topical ophthalmic 1-amino-3,5-dimethyladamantane hydrochloride (memantine) instillation was assessed by autoradiography. Briefly, albino and pigmented rabbits were dosed via topical ophthalmic instillation with a 0.74% (w/v) aqueous isotonic memantine solution having a pH of 7.4. After dosing, autoradiographic sections of the eye were acquired at 0.25, 0.5, 1, and 2 and 24 hours post dosing.

The autoradiographic data clearly demonstrated that memantine was present in the posterior sclera, choroid and/or retina after topical dosing. Further, a qualitative assessment of residence time was made from the data. The half-life of memantine in the posterior globe for both the albino and pigmented rabbits appears relatively long. The intensity of the autoradiography in the pigmented tissues indicates that memantine binds to the ocular melanin.

### EXAMPLE 9

Once a day topical dosing of 0.37% memantine HCl (w/v) borate buffered solution (0.242 mg/kg/day), was compared to daily oral dosing of memantine HCL, 1.76 mg/kg/day, in albino rabbits over seven days. The memantine levels in the retina obtained after topical administration were much higher than levels that were obtained from systemic dosing, 2,430 ng/mL for topical dosing and 100 ng/mL for oral dosing, respectively. Peak levels obtained by topical dosing far exceeded levels thought to be possible by oral dosing. Unfortunately, topical dosing of this formulation may lead to hyperemia, chemosis, and discomfort. Thus, topical dosing of this formulation is greatly limited.

### EXAMPLE 10

Tissue concentrations of memantine were determined after oral and topical dosing. Rabbits were dosed topically with 35 μl of a 0.1% (w/v) aqueous solution of radiolabeled memantine twice a day to both eyes for 7 days (equivalent to approximately 0.07 mg/kg/day). This composition is detailed in Table 1 as Composition 2. Another subset of rabbits was dosed orally with 2 mg/kg radiolabeled memantine for seven days. At the end of the dosing period ocular tissue concentrations were quantified. The retinal memantine concentrations from topical and oral dosing were essentially equivalent (108 ng/ml and 107 ng/ml, respectively). These data show that topically applied memantine at a 28 fold lower dose can achieve equivalent retinal concentrations to oral dosing.

### EXAMPLE 11

A toxicology study was conducted with a 1.5% (w/v) memantine HCl formulation dosed topically six times daily to Dutch Belted rabbits, TX99065. The study showed that 1.5% memantine HCl in a CMC-based formulation was well tolerated topically.

### EXAMPLE 12

A human oral clinical pharmacokinetic study showed that the mean plasma concentration of memantine following an oral dose of 20 mg per day was 95 ng/mL. Assuming a 31% protein binding and a retina-choroid concentration equivalent to the free-unbound plasma memantine concentration, the peak retinal level of memantine would be 0.30 μM.

A twice a day topical dosing of a 0.05% (w/v) and 0.25% (w/v) memantine solution in a CMC-based formulation resulted in peak retina levels of 0.30 μM and 1.4 μM without any signs of toxicity. Topical memantine in a CMC-based formulation is tolerated up to 1.5% w/v memantine. Clearly, topical memantine can achieve much higher retinal levels than can be obtained orally. In fact, topical memantine may be the only mechanism to achieve the effective retinal concentrations of about 1 to about 6 μM.

### EXAMPLE 13

A polyanionic polymer-containing composition according to one of Examples 1-6 is topically administered twice daily to a person suffering from an acute retinal injury for four (4) days. The patient is thereby provided with rapid treatment of his injury, for example, rapid reduction in one or more symptoms of his injury. Thereafter, the patient is orally administered a dose of memantine sufficient to provide a therapeutically effective amount of memantine with reduced side effects.

Thus, the patient receives rapid treatment of his acute retinal injury and continued treatment of his injury with reduced side effects. In contrast, if no topical administration is provided, oral administration would require a longer period of time, for example, on the order of about 1 week or about 2 weeks, before the therapeutic effects of memantine would be apparent.

While this invention has been described with respect to various specific examples and embodiments, it is to be understood that the invention is not limited thereto and that it can be variously practiced within the scope of the following claims.

1. A composition comprising an aqueous-based carrier and more than 0.1% w/v of an adamantane-based neuroprotective component solubilized in the carrier, the composition, when topically administered to an eye, being at least one of tolerated by the eye, non-toxic to the eye and effective to provide at least one of fewer side effects and reduced side effects relative to orally administering the adamantane-based neuroprotective component to the human or animal to provide the same concentration of the neuroprotective component in the retina of the eye.

2. The composition of claim 1 which, when topically administered to an eye, is tolerated by the eye and is non-toxic to the eye.

3. The composition of claim 1 which, when topically administered to an eye, causes at least one of substantially no irritation to the eye, substantially no discomfort, and substantially no pain.

4. The composition of claim 1 which, when topically administered to an eye, provides at least one of fewer side effects and reduced side effects relative to orally administering the adamantane-based neuroprotective component to the human or animal to provide the same concentration of the neuroprotective component in the retina of the eye.

5. The composition of claim 1 wherein the adamantane-based neuroprotective component comprises an adamantyl moiety and an amine moiety.

6. The composition of claim 5 wherein the adamantyl moiety is bonded directly to a nitrogen of the amine moiety.

7. The composition of claim 5 wherein the adamantane-based neuroprotective component further comprises a linking group bonded to the adamantyl moiety and the amine moiety.

8. The composition of claim 5 wherein the adamantyl moiety includes no substituent or at least one substituent.

9. The composition of claim 1 wherein, when topically administered to an eye, the adamantane-based neuroprotective component is effective to reduce the rate of ganglion cell loss as a result of a neurodegenerative disease in an eye.

10. The composition of claim 1 wherein the adamantane-based neuroprotective component is selected from the group consisting of amanitadine, remantadine, memantine and mixtures thereof.

11. The composition of claim 1 wherein the adamantane-based neuroprotective component is memantine.

12. The composition of claim 1 wherein the adamantane-based neuroprotective component is present in an amount in a range of more than 0.1% (w/v) to about 5% (w/v).

13. The composition of claim 1 wherein the adamantane-based neuroprotective component is present in an amount in a range of more than 0.1% (w/v) to about 1.5% (w/v).

14. The composition of claim 1 which further comprises a water soluble polymeric compatibility component present in an amount effective to enhance the ocular compatibility of the adamantane-based neuroprotective component relative to an identical composition without the compatibility component.

15. The composition of claim 14 wherein the compatibility component is a polyanionic polymeric component.

16. The composition of claim 14 wherein the compatibility component is present in an amount in a range of about 0.1% (w/v) to about 10% (w/v).

17. The composition of claim 14 wherein the compatibility component is selected from the group consisting of anionic cellulosic derivatives, hyaluronic acid, anionic starch derivatives, poly methacrylic acid, poly methacrylic acid derivatives, polyphospazene derivatives, poly aspartic acid, gelatin, alginic acid, alginic acid derivatives, poly acrylic acid, poly acrylic acid derivatives and mixtures thereof.

18. The composition of claim 14 wherein the compatibility component is carboxymethylcellulose.

19. A method for treating an eye of a human or animal comprising:
topically administering to an eye of a human or animal a composition comprising an aqueous-based carrier and an adamantane-based neuroprotective component solubilized in the carrier to provide a concentration of the neuroprotective component in the retina of the eye of at least about 0.4 μM, the topically administering step being at least one of tolerated by the eye, non-toxic to the eye, and effective to provide at least one of fewer side effects and reduced side effects relative to orally administering the adamantane-based neuroprotective component to the human or animal to provide the same concentration of the neuroprotective component in the retina of the eye.

20. The method of claim 19 wherein the administering step provides a concentration of the neuroprotective component in the retina of the eye of at least about 0.5 μM.

21. The method of claim 19 wherein the topically administering step provides at least one of fewer side effects and reduced side effects relative to orally administering the adamantane-based neuroprotective component to the human or animal to provide the same concentration of the neuroprotective component in the retina of the eye.

22. The method of claim 19 wherein the adamantane-based neuroprotective component comprises an adamantyl moiety and an amine moiety.

23. The method of claim 19 wherein the adamantane-based neuroprotective component is selected from the group consisting of amanitadine, remantadine, memantine and mixtures thereof.

24. The method of claim 19 wherein the adamantane-based neuroprotective component is memantine.

25. The method of claim 19 wherein the composition further comprises a water soluble polymeric compatibility component present in an amount effective to enhance the ocular compatibility of the adamantane-based neuroprotective component relative to an identical composition without the compatibility component.

26. The method of claim 25 wherein the compatibility component is a polyanionic polymeric component.

27. The method of claim 25 wherein the compatibility component is present in an amount in a range of about 0.1% (w/v) to about 10% (w/v).

28. The method of claim 25 wherein the compatibility component is selected from the group consisting of anionic cellulosic derivatives, hyaluronic acid, anionic starch derivatives, poly methacrylic acid, poly methacrylic acid derivatives, polyphospazene derivatives, poly aspartic acid, gelatin, alginic acid, alginic acid derivatives, poly acrylic acid, poly acrylic acid derivatives and mixtures thereof.

29. The method of claim 25 wherein the compatibility component is carboxymethylcellulose.

30. A method for treating an eye of a human or animal comprising:
topically administering to an eye of a human or animal a first composition comprising an aqueous-based carrier and a first adamantane-based neuroprotective component solubilized in the carrier; and
orally administering to the human or animal a second composition comprising a second adamantane-based neuroprotective component.

31. The method of claim 30 wherein the orally administering step occurs after the topically administering step.

32. The method of claim 30 wherein the topically administering step provides a therapeutically effective amount of an adamantane-based neuroprotective component to a retina of the eye more rapidly than an identical method without the topically administering step.

33. The method of claim 30 wherein the topically administering step provides a concentration of at least about 0.3 μM of an adamantine-based neuroprotective component in a retina of an eye.

34. The method of claim 30 which is employed to treat an acute indication.

35. The method of claim 30 which is employed as a prophylaxis for the eye.

36. The method of claim 30 wherein the first adamantine-based neuroprotective component and the second adamantine-based neuroprotective component are the same or different.

37. The method of claim 30 wherein each of the first and second adamantane-based neuroprotective components is independently selected from the group consisting of amanitadine, remantadine, memantine and mixtures thereof.

38. The method of claim 30 wherein the first composition further comprises a water soluble polymeric compatibility component present in an amount effective to enhance the ocular compatibility of the first adamantane-based neuroprotective component relative to an identical composition without the compatibility component.

39. The method of claim 38 wherein the compatibility component is a polyanionic polymeric component.

40. The method of claim 38 wherein the compatibility component is selected from the group consisting of anionic cellulosic derivatives, hyaluronic acid, anionic starch derivatives, poly methacrylic acid, poly methacrylic acid derivatives, polyphospazene derivatives, poly aspartic acid, gelatin, alginic acid, alginic acid derivatives, poly acrylic acid, poly acrylic acid derivatives and mixtures thereof.
